(19) United States
(12) Patent Application Publication
(10) Pub. No.: US 2023/0129891 A1
(43) Pub. Date: Apr. 27, 2023

(54) TREATMENT APPARATUS FOR AN EYE TREATMENT, METHOD, COMPUTER PROGRAM, COMPUTER-READABLE MEDIUM AND STORAGE DEVICE

(71) Applicant: SCHWIND eye-tech-solutions GmbH, Kleinostheim (DE)

(72) Inventors: Samuel ARBA MOSQUERA, Aschaffenburg (DE); Luise KRÜGER, Aschaffenburg (DE); Pascal NAUBEREIT, Aschaffenburg (DE); Shwetabh VERMA, Aschaffenburg (DE); Thomas WENDLER, Stockstadt (DE)

(21) Appl. No.: 18/048,277

(22) Filed: Oct. 20, 2022

(30) Foreign Application Priority Data
Oct. 21, 2021 (DE) 10 2021 127 402.8

(51) Int. Cl. A61F 9/008

(52) U.S. Cl. CPC A61F 9/008; A61F 2009/00872; A61F 2009/00897

(57) ABSTRACT

The invention relates to a treatment apparatus (1) for an eye treatment, at least comprising: at least one laser beam source (3) configured for emission of a laser beam (2), at least one beam exit device (9), which is configured to direct the laser beam (2) to an eye (11) to be treated, at least one transfer optics (6), which is configured to feed the laser beam (2) along a respective optical path (21) to the at least one beam exit device (9), and a control device (14), which is configured to retrieve a predetermined eye treatment configuration (15) from a storage device (13), on which multiple eye treatment configurations (12) are stored, to adjust the at least one laser beam source (3) for generating the laser beam (2) of a pulse duration (4) to be adjusted according to the predetermined eye treatment configuration (15) of the eye treatment configurations (12), and to adjust the at least one transfer optics (6) for providing a predetermined numerical aperture (10) of the transfer optics (6) according to the predetermined eye treatment configuration (15).

### FIELD

The present invention relates to a treatment apparatus for an eye treatment. In addition, the invention relates to a method for controlling the treatment apparatus, to a computer program, to a computer-readable medium and to a storage device.

### BACKGROUND

Treatment apparatuses and methods for controlling ophthalmological lasers for correcting an optical visual disorder and/or pathologically and/or unnaturally altered areas of the cornea are known in the prior art. Therein, a pulsed laser and a beam focusing device can for example be formed such that laser pulses cause a photodisruption and/or photoablation in a focus located within the organic tissue to remove a tissue, in particular a tissue lenticule, from the cornea.

According to the current prior art, it is usual to provide specific treatment apparatuses for respective treatment methods, which are optimized for performing the respective treatment method. Herein, it is disadvantageous that for performing different treatment methods, the respective treatment apparatuses thus have to be provided. This results in higher cost and an increased space requirement. The treatment apparatuses configured for different treatment methods in particular differ in their laser beam sources, their transfer optics and their exit devices. This is due to the fact that the different eye treatment methods require respective pulse durations of the laser beam, respective wavelengths of the laser beam, respective numerical apertures of the transfer optics and special exit devices.

A simple combination of multiple of the treatment apparatuses to a new treatment apparatus would continue to require an increased space requirement.

The following treatment apparatuses for an eye treatment are known. US 2005/0085800 A1 discloses an apparatus and a method for refractive laser surgery. With the aid of a laser beam source, an fs-impulse laser beam is generated. A second laser beam source generates a UV laser beam. A shared scanner apparatus uses the fs-impulse laser beam and the UV laser beam for scanning a target object. U.S. Pat. No. 9,974,690 B2 discloses an apparatus and a method for laser in-situ keratomileusis (LASIK). The apparatus includes a first laser radiation source for generating first laser radiation pulses; first means for guiding and shaping the first laser radiation pulses; a second laser radiation source for generating second laser radiation pulses; second means for guiding and shaping the second laser radiation pulses; a control device comprising: a first treatment program for controlling the first means and the first laser radiation pulses for the purpose of generating a cut in the cornea, wherein the first treatment program generates regular corneal surface structures; a second treatment program for controlling the second means and the second laser radiation pulses for the purpose of reshaping the cornea and changing its imaging characteristics; and a third treatment program for controlling the second means and the second laser radiation pulses for the purpose of eliminating the previously mentioned regular structures.

### SUMMARY

The invention is based on the object to provide a treatment apparatus, which is configured for performing various treatment methods.

This object is solved by a treatment apparatus, method, computer program, computer-readable medium and storage device with features as described herein. Advantageous configurations with convenient developments of the invention are also specified herein, wherein advantageous configurations of each inventive aspect are to be regarded as advantageous configurations of the respectively other inventive aspects.

A first aspect of the invention relates to a treatment apparatus for an eye treatment. It is provided that the treatment apparatus comprises at least one laser beam source, which is configured for emission of a laser beam. The treatment apparatus comprises at least one beam exit device, which is configured to direct the laser beam to an eye to be treated. The at least one beam exit device is thus formed as a patient interface. The treatment apparatus comprises at least one transfer optics, which is configured to feed the laser beam along a respective optical path to the at least one beam exit device. In other words, the treatment apparatus includes the at least one transfer optics, which is provided to feed the laser beam emitted by the laser beam source along the respective optical path to the at least one beam exit device. The treatment apparatus includes a control device, which is configured to retrieve a predetermined eye treatment configuration from a storage device, on which multiple eye treatment configurations are stored. The control device is configured to adjust the at least one laser beam source for generating the laser beam of a pulse duration to be adjusted according to the predetermined eye treatment configuration of the eye treatment configurations and to adjust the at least one transfer optics for providing a numerical aperture of the transfer optics to be adjusted according to the predetermined eye treatment configuration. If the treatment apparatus comprises at least two of the laser beam sources, which for example differ in their pulse durations, the control device can be configured to select and adjust the laser beam source, which is configured for generating the laser beam with the pulse duration to be adjusted. If the treatment apparatus comprises at least two of the transfer optics, which for example differ in their numerical apertures, the control device can be configured to select and adjust the transfer optics, which is configured for providing the numerical aperture to be adjusted.

In other words, the treatment apparatus includes the control device. The control device is configured to read the predetermined eye treatment configuration out of the storage device. The storage device can for example include a hard disk or a further storage medium, wherein the multiple eye treatment configurations are stored on the storage device. The eye treatment configurations can be files, which can preset respective pulse durations to be provided by the laser beam source and numerical apertures to be provided by the transfer optics. The storage device can be arranged in the treatment apparatus or be situated outside of it. The predetermined eye treatment configuration of the eye treatment configurations can be one of the eye treatment configurations, which can be associated with an eye treatment to be performed by the treatment apparatus. The selection of the predetermined eye treatment configuration can for example have been effected by means of a predetermined user input in the treatment apparatus. The control device is configured to adjust the laser beam source to generate the laser beam with the pulse duration to be adjusted, which is preset in the predetermined eye treatment configuration. The term of the adjustment can also include a selection of a laser beam source if the treatment apparatus includes multiple laser beam sources, which are configured for outputting laser beams of an invariable pulse duration. The control device is configured to adjust the transfer optics to adjust the predetermined numerical aperture of the transfer optics according to the predetermined eye treatment configuration. By the invention, the advantage arises that the treatment apparatus is configured to perform eye treatments, which can differ from each other in the pulse duration and numerical aperture to be adjusted.

The invention also includes developments, by which additional advantages arise.

A development of the invention provides that the treatment apparatus includes a laser beam source. In other words, the treatment apparatus comprises a single laser beam source, which is configured to emit the laser beam. By the development, the advantage arises that a single laser beam source is provided for performing different treatment methods. Preferably, the laser beam source can be an excimer laser, a gas laser, a solid-state laser, a laser diode, a fiber laser, preferably with a fiber oscillator and/or a fiber amplifier.

A development of the invention provides that the control device is configured to adjust the at least one laser beam source for generating the laser beam of a wavelength to be adjusted according to the predetermined eye treatment configuration. In other words, the control device is provided to adjust the at least one laser beam source to effect the emissions of the laser beam with the wavelength to be adjusted. Therein, the wavelength to be adjusted is set by the predetermined eye treatment configuration like the numerical aperture to be adjusted and the pulse duration to be adjusted. The laser beam source can be formed for allowing the adjustment of the wavelength as a tunable laser. The at least one laser beam source can be configured to adapt the wavelength via harmonic generation. Thereby, the wavelength can be halved; split into thirds or generally adapted by a factor of 1/n, wherein n is an integer number. For example, N can have values up to n=6 and thus for example be 2, 3, 4, 5, 6. The laser beam source can be configured to also optically shift the wavelength. For example, this can be effected by means of Raman shift. Hereto, the laser beam source can comprise at least one suitable non-linear medium, which allows this conversion. The conversion can for example be coherent anti-Stokes Raman scattering and/or stimulated anti-Stokes Raman scattering. If the treatment apparatus comprises at least two of the laser beam sources, the control device can be configured to select and control the laser beam source, which is configured for outputting the laser beam with the respective wavelength. By the development, the advantage arises that the treatment apparatus can perform different eye treatments even if they require different wavelengths of the laser beam.

A development of the invention provides that in at least one first eye treatment configuration of the eye treatment configurations, the pulse duration of the laser beam to be adjusted is between 100 fs and 1 ps and the numerical aperture of the transfer optics to be adjusted is between 0.3 and 1.0. In other words, the eye treatment configurations include the at least one first eye treatment configuration, which presets the pulse duration of the laser beam to be adjusted with a value between 100 fs and 1 ps. For example, the value of the pulse duration of the laser beam to be adjusted can be 100 fs, 150 fs, 200 fs, 250 fs, 300 fs, 350 fs, 400 fs, 450 fs, 500 fs, 550 fs, 600 fs, 650 fs, 700 fs, 750 fs, 800 fs, 850 fs, 900 fs, 950 fs, 1000 fs. The at least one first eye treatment configuration presets a value between 0.3 and 1.0, in particular a value between 0.4 and 0.6, for the numerical aperture to be adjusted. For example, the value of the numerical aperture to be adjusted can be 0.30, 0.31, 0.32, 0.33, 0.34, 0.35, 0.36, 0.37, 0.38, 0.39, 0.40, 0.41, 0.42, 0.43, 0.44, 0.45, 0.46, 0.47, 0.48, 0.49, 0.50, 0.51, 0.52, 0.53, 0.54, 0.55, 0.56, 0.57, 0.58, 0.59, 0.60, 0.61, 0.62, 0.63, 0.64, 0.65, 0.66, 0.67, 0.68, 0.69, 0.70, 0.71, 0.72, 0.73, 0.74, 0.75, 0.76, 0.77, 0.78, 0.79, 0.80, 0.81, 0.82, 0.83, 0.84, 0.85, 0.86, 0.87, 0.88, 0.89, 0.90, 0.91, 0.92, 0.93, 0.94, 0.95, 0.96, 0.97, 0.98, 0.99, 1.00. By the development, the advantage arises that an eye treatment configuration is provided with the at least one first eye treatment configuration, which presets values of the pulse duration of the laser beam and the numerical aperture of the transfer optics to be adjusted, which are for example required for performing corneal cuts. Thereby, it is possible to perform corneal cuts by means of the treatment apparatus.

A development of the invention provides that in the at least one first eye treatment configuration, the wavelength of the laser beam to be adjusted is between 380 nm and 1200 nm. In other words, it is provided that a value for the wavelength of the laser beam to be adjusted is preset by the at least one first eye treatment configuration, which is between 380 nm and 1200 nm. For example, the value of the wavelength of the laser beam to be adjusted can be 380 nm, 390 nm, 400 nm, 410 nm, 420 nm, 430 nm, 440 nm, 450 nm, 460 nm, 470 nm, 480 nm, 490 nm, 500 nm, 510 nm, 520 nm, 530 nm, 540 nm, 550 nm, 560 nm, 570 nm, 580 nm, 590 nm, 600 nm, 610 nm, 620 nm, 630 nm, 640 nm, 650 nm, 660 nm, 670 nm, 680 nm, 690 nm, 700 nm, 710 nm, 720 nm, 730 nm, 740 nm, 750 nm, 760 nm, 770 nm, 780 nm, 790 nm, 800 nm, 810 nm, 820 nm, 830 nm, 840 nm, 850 nm, 860 nm, 870 nm, 880 nm, 890 nm, 900 nm, 910 nm, 920 nm, 930 nm, 940 nm, 950 nm, 960 nm, 970 nm, 980 nm, 990 nm, 1000 nm, 1010 nm, 1020 nm, 1030 nm, 1040 nm, 1050 nm, 1060 nm, 1070 nm, 1080 nm, 1090 nm, 1100 nm, 1110 nm, 1120 nm, 1130 nm, 1140 nm, 1150 nm, 1160 nm, 1170 nm, 1180 nm, 1190 nm, 1200 nm.

A development of the invention provides that in at least one second eye treatment configuration of the eye treatment configurations, the pulse duration of the laser beam to be adjusted is between 10 fs and 150 fs and the numerical aperture of the transfer optics to be adjusted is between 0.18 and 0.35. In other words, the eye treatment configurations include the at least one second eye treatment configuration, which presets the pulse duration of the laser beam to be adjusted with a value between 10 fs and 150 fs. For example, the value of the pulse duration to be adjusted can be 10 fs, 20 fs, 30 fs, 40 fs, 50 fs, 60 fs, 70 fs, 80 fs, 90 fs, 100 fs, 110 fs, 120 fs, 130 fs, 140 fs, 150 fs. The at least one second eye treatment configuration presets a value between 0.18 and 0.35 for the numerical aperture to be adjusted. For example, the value of the numerical aperture to be adjusted can be 0.18, 0.19, 0.20, 0.21, 0.22, 0.23, 0.24, 0.25, 0.26, 0.27, 0.28, 0.29, 0.30, 0.31, 0.32, 0.33, 0.34, 0.35. By the development, the advantage arises that an eye treatment configuration is provided with the at least one second eye treatment configuration, which presets values of the pulse duration of the laser beam and the numerical aperture of the transfer optics to be adjusted, which are for example required for performing eye treatment methods for laser-induced refractive index change (LIRIC) and/or eye treatment methods for laser-assisted refractive index shaping (RIS). Thereby, it is possible to perform the said eye treatment methods by means of the treatment apparatus.

A development of the invention provides that in the at least one second eye treatment configuration, the wavelength of the laser beam to be adjusted is between 380 nm and 1200 nm. In other words, it is provided that a value for the wavelength of the laser beam to be adjusted is preset by the at least one second eye treatment configuration, which is between 380 nm and 1200 nm. For example, the value of the wavelength of the laser beam to be adjusted can be 380 nm, 390 nm, 400 nm, 410 nm, 420 nm, 430 nm, 440 nm, 450 nm, 460 nm, 470 nm, 480 nm, 490 nm, 500 nm, 510 nm, 520 nm, 530 nm, 540 nm, 550 nm, 560 nm, 570 nm, 580 nm, 590 nm, 600 nm, 610 nm, 620 nm, 630 nm, 640 nm, 650 nm, 660 nm, 670 nm, 680 nm, 690 nm, 700 nm, 710 nm, 720 nm, 730 nm, 740 nm, 750 nm, 760 nm, 770 nm, 780 nm, 790 nm, 800 nm, 810 nm, 820 nm, 830 nm, 840 nm, 850 nm, 860 nm, 870 nm, 880 nm, 890 nm, 900 nm, 910 nm, 920 nm, 930 nm, 940 nm, 950 nm, 960 nm, 970 nm, 980 nm, 990 nm, 1000 nm, 1010 nm, 1020 nm, 1030 nm, 1040 nm, 1050 nm, 1060 nm, 1070 nm, 1080 nm, 1090 nm, 1100 nm, 1110 nm, 1120 nm, 1130 nm, 1140 nm, 1150 nm, 1160 nm, 1170 nm, 1180 nm, 1190 nm, 1200 nm.

A development of the invention provides that in at least one third eye treatment configuration of the eye treatment configurations, the pulse duration of the laser beam to be adjusted is between 20 fs and 200 fs and the numerical aperture of the transfer optics to be adjusted is between 0.05 and 0.25. In other words, the eye treatment configurations include the at least one third eye treatment configuration, which presets the pulse duration of the laser beam to be adjusted with a value between 20 fs and 200 fs. For example, the value of the pulse duration to be adjusted can be 20 fs, 30 fs, 40 fs, 50 fs, 60 fs, 70 fs, 80 fs, 90 fs, 100 fs, 110 fs, 120 fs, 130 fs, 140 fs, 150 fs, 160 fs, 170 fs, 180 fs, 190 fs, 200 fs. The at least one third eye treatment configuration presets a value between 0.05 and 0.25 for the numerical aperture to be adjusted. For example, the value of the numerical aperture to be adjusted can be 0.05, 0.06, 0.07, 0.08, 0.09, 0.10, 0.11, 0.12, 0.13, 0.14, 0.15, 0.16, 0.17, 0.18, 0.19, 0.20, 0.21, 0.22, 0.23, 0.24, 0.25. By the development, the advantage arises that an eye treatment configuration is provided with the at least one third eye treatment configuration, which presets values of the pulse duration of the laser beam and the numerical aperture of the transfer optics to be adjusted, which are for example required for performing eye treatment methods for corneal cross linking (CXL). Thereby, it is possible to perform eye treatment methods for corneal cross linking by means of the treatment apparatus.

A development of the invention provides that in the at least one third eye treatment configuration, the wavelength of the laser beam to be adjusted is between 360 nm and 950 nm. In other words, it is provided that a value for the wavelength of the laser beam to be adjusted is preset by the at least one third eye treatment configuration, which is between 360 nm and 950 nm. For example, the value of the wavelength of the laser beam to be adjusted can be 360 nm, 370 nm, 380 nm, 390 nm, 400 nm, 410 nm, 420 nm, 430 nm, 440 nm, 450 nm, 460 nm, 470 nm, 480 nm, 490 nm, 500 nm, 510 nm, 520 nm, 530 nm, 540 nm, 550 nm, 560 nm, 570 nm, 580 nm, 590 nm, 600 nm, 610 nm, 620 nm, 630 nm, 640 nm, 650 nm, 660 nm, 670 nm, 680 nm, 690 nm, 700 nm, 710 nm, 720 nm, 730 nm, 740 nm, 750 nm, 760 nm, 770 nm, 780 nm, 790 nm, 800 nm, 810 nm, 820 nm, 830 nm, 840 nm, 850 nm, 860 nm, 870 nm, 880 nm, 890 nm, 900 nm, 910 nm, 920 nm, 930 nm, 940 nm, 950 nm.

A development of the invention provides that in at least one fourth eye treatment configuration of the eye treatment configurations, the pulse duration of the laser beam to be adjusted is between 300 fs and 1 ps and the numerical aperture of the transfer optics to be adjusted is between 0.05 and 0.15. In other words, the eye treatment configurations include the at least one fourth eye treatment configuration, which presets the pulse duration of the laser beam to be adjusted with a value between 300 fs and 1 ps. For example, the value of the pulse duration to be adjusted can be 300 fs, 350 fs, 400 fs, 450 fs, 500 fs, 550 fs, 600 fs, 650 fs, 700 fs, 750 fs, 800 fs, 850 fs, 900 fs, 950 fs, 1000 fs. The at least one fourth eye treatment configuration presets a value between 0.05 and 0.15 for the numerical aperture to be adjusted. For example, the value of the numerical aperture to be adjusted can be 0.05, 0.06, 0.07, 0.08, 0.09, 0.10, 0.11, 0.12, 0.13, 0.14, 0.15. By the development, the advantage arises that an eye treatment configuration is provided with the at least one fourth eye treatment configuration, which presets values of the pulse duration of the laser beam and the numerical aperture of the transfer optics to be adjusted, which are for example required for performing eye treatment methods for treating presbyopia or for performing eye treatment methods for treating vitreous body destructions, in particular vitreous floaters. Thereby, it is possible to perform the said eye treatment methods by means of the treatment apparatus.

A development of the invention provides that in the at least one fourth eye treatment configuration, the wavelength of the laser beam to be adjusted is between 760 nm and 1500 nm. In other words, it is provided that a value for the wavelength of the laser beam to be adjusted is preset by the at least one fourth eye treatment configuration, which is between 760 nm and 1500 nm. For example, the value of the wavelength of the laser beam to be adjusted can be 760 nm, 770 nm, 780 nm, 790 nm, 800 nm, 810 nm, 820 nm, 830 nm, 840 nm, 850 nm, 860 nm, 870 nm, 880 nm, 890 nm, 900 nm, 910 nm, 920 nm, 930 nm, 940 nm, 950 nm, 960 nm, 970 nm, 980 nm, 990 nm, 1000 nm, 1010 nm, 1020 nm, 1030 nm, 1040 nm, 1050 nm, 1060 nm, 1070 nm, 1080 nm, 1090 nm, 1100 nm, 1110 nm, 1120 nm, 1130 nm, 1140 nm, 1150 nm, 1160 nm, 1170 nm, 1180 nm, 1190 nm, 1200 nm, 1210 nm, 1220 nm, 1230 nm, 1240 nm, 1250 nm, 1260 nm, 1270 nm, 1280 nm, 1290 nm, 1300 nm, 1310 nm, 1320 nm, 1330 nm, 1340 nm, 1350 nm, 1360 nm, 1370 nm, 1380 nm, 1390 nm, 1400 nm, 1410 nm, 1420 nm, 1430 nm, 1440 nm, 1450 nm, 1460 nm, 1470 nm, 1480 nm, 1490 nm, 1500 nm.

A development of the invention provides that in at least one fifth eye treatment configuration of the eye treatment configurations, the pulse duration of the laser beam to be adjusted is between 100 fs and 1 ps and the numerical aperture of the transfer optics to be adjusted is between 0 and 0.1. In other words, the eye treatment configurations include the at least one fifth eye treatment configuration, which presets the pulse duration of the laser beam to be adjusted with a value between 100 fs and 1 ps. For example, the value of the pulse duration to be adjusted can be 100 fs, 150 fs, 200 fs, 250 fs, 300 fs, 350 fs, 400 fs, 450 fs, 500 fs, 550 fs, 600 fs, 650 fs, 700 fs, 750 fs, 800 fs, 850 fs, 900 fs, 950 fs, 1000 fs. The at least one fifth eye treatment configuration presets a value between 0 and 0.1 for the numerical aperture to be adjusted. For example, the value of the numerical aperture to be adjusted can be 0.00, 0.01, 0.02, 0.03, 0.04, 0.05, 0.06, 0.07, 0.08, 0.09, 0.10. By the development, the advantage arises that an eye treatment configuration is provided with the at least one fifth eye treatment configuration, which presets values of the pulse duration of the laser beam and the numerical aperture of the transfer optics to be adjusted, which are for example used for performing tissue ablations on the cornea. Thereby, it is possible to perform eye treatment methods for tissue ablations on the cornea by means of the treatment apparatus.

A development of the invention provides that in the at least one fifth eye treatment configuration, the wavelength of the laser beam to be adjusted is between 150 nm and 230 nm. In other words, it is provided that a value for the wavelength of the laser beam to be adjusted is preset by the at least one fifth eye treatment configuration, which is between 150 nm and 230 nm. For example, the value of the wavelength of the laser beam to be adjusted can be 150 nm, 155 nm, 160 nm, 165 nm, 170 nm, 175 nm, 180 nm, 185 nm, 190 nm, 195 nm, 200 nm, 205 nm, 210 nm, 215 nm, 220 nm, 225 nm, 230 nm.

A development of the invention provides that the treatment apparatus includes at least two of the beam exit devices and the control device is configured to adjust the at least one transfer optics for feeding the laser beam to a beam exit device to be adjusted according to the predetermined eye treatment configuration. In other words, the treatment apparatus comprises multiple of the beam exit devices. The control device of the treatment apparatus is configured to adjust the at least one transfer optics to feed the laser beam to the beam exit device to be adjusted according to the predetermined eye treatment configuration. By the development, the advantage arises that multiple of the beam exit devices are provided, which can be specialized in different ones of the eye treatment methods. Therein, in the respective eye treatment configurations, the beam exit devices to be adjusted, which are provided for performing the respective eye treatment method, are predetermined.

A development of the invention provides that the control device is configured to adjust the at least one laser beam source for generating the laser beam of an energy, to be adjusted, of a laser pulse of the laser beam according to the predetermined eye treatment configuration. In other words, it is provided that the control device adjusts the at least one laser beam source in order that it outputs at least one laser pulse, which has a predetermined energy. If the treatment apparatus comprises multiple of the laser beam sources, the control device can ascertain and adjust that one of the laser beam sources, which is configured for providing the energy. The energy to be adjusted can be preset by the eye treatment configuration.

A second aspect of the invention relates to a method for controlling the treatment apparatus by the control device. The method includes at least certain steps. In at least one of the steps, adjusting the at least one laser beam source for generating the laser beam of the pulse duration to be adjusted according to the preset eye treatment configuration is effected by the control device. In other words, in the step, the pulse duration to be adjusted is adjusted to the value in the at least one laser beam source by the control device as it is preset in the preset eye treatment configuration. In a further one of the steps, adjusting the at least one transfer optics for providing the numerical aperture to be adjusted according to the preset eye treatment configuration is effected by the control device. In other words, in a step of the method, it is provided that the at least one transfer optics is adjusted to the numerical aperture to be adjusted by the control device as it is preset in the preset eye treatment configuration.

A development of the invention provides that the method includes at least the step, which includes adjusting the at least one laser beam source for generating the laser beam of the wavelength to be adjusted according to the preset eye treatment configuration by the control device. In other words, it is provided in the step that the at least one laser beam source is adjusted by the control device to provide the laser beam of the wavelength to be adjusted according to the preset eye treatment configuration.

A development of the invention provides that the method includes at least the step, which includes adjusting the at least one transfer optics for feeding the laser beam to the beam exit device to be adjusted according to the predetermined eye treatment configuration. In other words, it is provided that the at least one transfer optics is adjusted by the control device to direct the laser beam to the beam exit device to be adjusted as it is preset in the predetermined eye treatment configuration.

A development of the invention provides that the method includes at least the step, which includes adjusting the at least one laser beam source for generating the laser beam of the energy, to be adjusted, of a laser pulse of the laser beam according to the predetermined eye treatment configuration. In other words, it is provided in the step that the at least one laser beam source is adjusted by the control device to provide the laser beam of the energy to be adjusted according to the preset eye treatment configuration.

Further features and the advantages thereof can be taken from the descriptions of the first inventive aspect, wherein advantageous configurations of each inventive aspect are to be regarded as advantageous configurations of the respectively other inventive aspect.

A third aspect of the invention relates to a computer program including commands, which cause the treatment apparatus according to the first inventive aspect to execute the method steps according to the second inventive aspect.

Further features and the advantages thereof can be taken from the descriptions of the first and the second inventive aspect, wherein advantageous configurations of each inventive aspect are to be regarded as advantageous configurations of the respectively other inventive aspect.

A fourth aspect of the invention relates to a computer-readable medium, on which the computer program according to the third inventive aspect is stored. Further features and the advantages thereof can be taken from the descriptions of the first to third inventive aspects, wherein advantageous configurations of each inventive aspect are to be regarded as advantageous configurations of the respectively other inventive aspect.

Further features and the advantages thereof can be taken from the descriptions of the first, the second and the third inventive aspect, wherein advantageous configurations of each inventive aspect are to be regarded as advantageous configurations of the respectively other inventive aspect.

A fifth aspect of the invention relates to a storage device, on which the eye treatment configurations are stored. The storage device can be configured as a volatile data memory, for example as a dynamic random access memory, DRAM, or static random access memory, SRAM, or as a non-volatile data memory, for example as a read-only memory, ROM, as a programmable read-only memory, PROM, as an erasable read-only memory, EPROM, as an electrically erasable read-only memory, EEPROM, as a flash memory or flash EEPROM, as a ferroelectric random access memory, FRAM, as a magnetoresistive random access memory, MRAM, or as a phase-change random access memory, PCRAM.

Further features and the advantages thereof can be taken from the descriptions of the first, the second, the third and the fourth inventive aspect, wherein advantageous configurations of each inventive aspect are to be regarded as advantageous configurations of the respectively other inventive aspect.

### BRIEF DESCRIPTION OF THE DRAWINGS

Further features are apparent from the claims, the figures and the description of figures. The features and feature combinations mentioned above in the description as well as the features and feature combinations mentioned below in the description of figures and/or shown in the figures alone are usable not only in the respectively specified combination, but also in other combinations without departing from the scope of the invention. Thus, implementations are also to be considered as encompassed and disclosed by the invention, which are not explicitly shown in the figures and explained but arise from and can be generated by separated feature combinations from the explained implementations. Implementations and feature combinations are also to be considered as disclosed, which thus do not comprise all of the features of an originally formulated independent claim. Moreover, implementations and feature combinations are to be considered as disclosed, in particular by the implementations set out above, which extend beyond or deviate from the feature combinations set out in the relations of the claims.

FIG. 1 depicts a schematic representation of an embodiment of a treatment apparatus.

FIG. 2 depicts a schematic representation of a further embodiment of a treatment apparatus.

FIG. 3 depicts a schematic representation of a further embodiment of a treatment apparatus.

FIG. 4 depicts a schematic representation of a sequence of a method for controlling a treatment apparatus.

### DETAILED DESCRIPTION

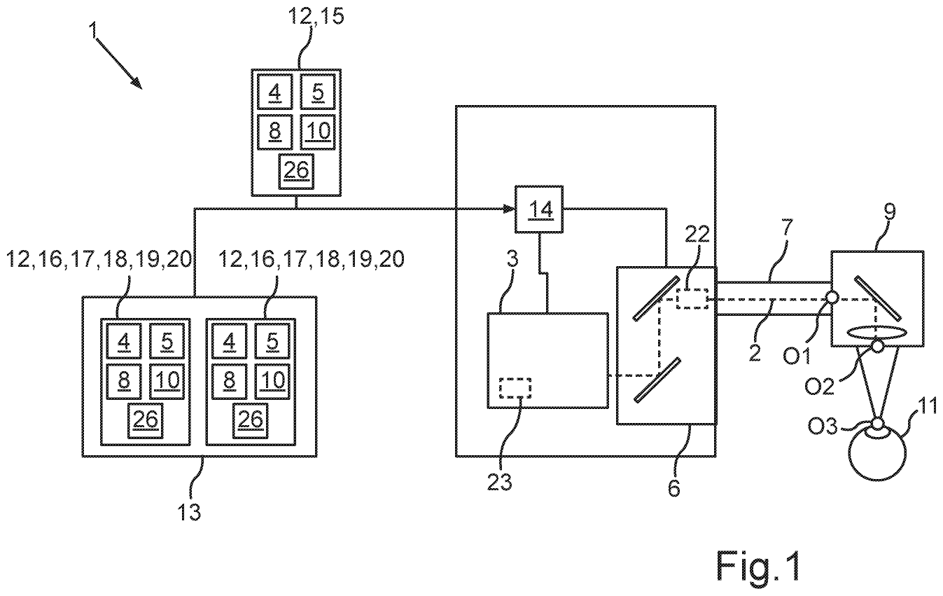

FIG. 1 shows a schematic representation of an embodiment of a treatment apparatus. The treatment apparatus 1 can be configured for performing predetermined eye treatment methods by means of a laser beam 2. In particular, the eye treatment methods can include corneal cuts, eye treatment methods for laser-induced refractive index change (URIC); eye treatment methods for laser-assisted refractive index shaping (RIS); eye treatment methods for corneal cross linking (CXL); eye treatment methods for treating presbyopia; eye treatment methods for treating vitreous body destructions, in particular vitreous floaters; and eye treatment methods for tissue ablations on the cornea.

The treatment apparatus 1 can comprise exactly one or at least one laser beam source 3 for emission of the laser beam 2.

The treatment apparatus 1 can comprise transfer optics 6, which is configured to feed the laser beam 2 emitted by the at least one laser beam source 3 along a respective optical path 7 to a beam exit device 8, to be adjusted, of at least one beam exit device 9 of the treatment apparatus 1. The transfer optics 6 can for example include mirrors, light guides and/or shutters as elements, of which individual ones of the elements can be adjusted by the control device 14.

The beam exit device 9 can be formed as a so-called patient interface and for example comprise an apparatus for fixing an eye 11 to be treated. The respective beam exit device 9 can comprise a scanner, which can be configured to guide the laser beam 2 along predetermined paths towards the eye 11.

The at least one transfer optics 6 can be configured to provide a predetermined numerical aperture 10. The numerical aperture 10 to be adjusted can for example relate to the numerical aperture, which describes to a point O1 at an entry point of the beam exit device 8 to be adjusted, to a point O2 at an exit point of the laser beam 2 from the beam exit device 8 to be adjusted or to a point O3 in a predetermined depth in the eye 11. The numerical aperture 10 to be adjusted can in particular relate to a point O3, which can be arranged on a top side of the cornea.

The eye treatment methods can make respective demands to the laser beam 2. Depending on an eye treatment method to be performed, it can thus be required to generate the laser beam 2 with a pulse duration 4 to be adjusted, which can be required for the respective eye treatment method. It can also be required to emit the laser beam 2 with a wavelength 5 to be adjusted.

For performing the respective eye treatment methods, it can be required that the respective pulse duration 4 to be adjusted and the respective numerical aperture 10 to be adjusted have to be provided by the treatment apparatus 1. Further, an adjustment of the respective wavelength 5 to be adjusted and of an energy 26 of a laser pulse to be adjusted can also be required. The pulse duration 4 to be adjusted, the wavelength 5 to be adjusted, the numerical aperture 10 to be adjusted and the beam exit device 8 to be adjusted can be preset in respective eye treatment configurations 12, which can be stored on a storage device 13. The eye treatment configurations 12 can be matched with the respective eye treatment methods.

The treatment apparatus 1 can comprise a control device 14, which can be configured to read at least one of the eye treatment configurations 12 out of the storage device 13 as a predetermined eye treatment configuration 15. The selection of the predetermined eye treatment configuration 15 can for example be effected depending on a predetermined user input, which can be received by the control device 14. The storage device can be installed in the treatment apparatus 1 or be arranged apart from it. The storage device can for example include a hard disk or a flash memory. The control device 14 can be configured to open the predetermined eye treatment configuration 15 and to read out the pulse duration 4 to be adjusted, the wavelength 5 to be adjusted, the beam exit device 8 to be adjusted, the energy 26 of a laser pulse to be adjusted and the numerical aperture 10 to be adjusted, which are preset in the predetermined eye treatment configuration 15. The control device 14 can be configured to adjust the pulse duration 4 to be adjusted and/or the wavelength 5 to be adjusted and/or the energy 26 of a laser pulse to be adjusted in the at least one laser beam source 3. Thereby, it can be allowed that the laser beam 2 is emitted by the at least one laser beam source 3, which can have the pulse duration 4 to be adjusted, the energy 26 of a laser pulse to be adjusted and the wavelength 5 to be adjusted. The control device 14 can be configured to adjust the transfer optics 6 according to the numerical aperture 10 to be adjusted according to the predetermined eye treatment configuration 15 in order that the numerical aperture 10 to be adjusted can be provided by the transfer optics 6. The control device 14 can also be configured to control the transfer optics 6 according to the beam exit device 8 to be adjusted in order that the laser beam 2 of the beam exit device 8 to be adjusted is fed to the at least one beam exit device 9.

For adjusting the numerical aperture 10 to be adjusted, the control device 14 can for example be configured to adjust an NA adapter 22 of the transfer optics 6, which can comprise a movable shutter, a variable telescope and/or a beam expander, such that the numerical aperture 10 to be adjusted is provided by the transfer optics 6. The control device 14 can be configured to provide the pulse duration 4 to be adjusted by a pre-chirp of the laser beam source 3 and/or via a compressor of the laser beam source 3 and/or via spectral broadening in a fiber laser.

For adjusting the beam exit device 8 to be adjusted, the transfer optics can include a beam exit selector 25, wherein it can comprise a movable mirror to direct the laser beam to the beam exit device 8 to be adjusted.

The control device 14 can be configured to adjust the wavelength 5 of the laser beam to be adjusted. The at least one laser beam source 3 can for example include a TI:SA laser, a supercontinuum laser or an optical parametric generator (OPG), an optical parametric oscillator (OPO), an optical parametric amplifier (OPA), a noncollinear OPA (NOPA), or a multi-pass OPA. This can allow the provision of laser beams 2 of different wavelengths 5 to be adjusted. Hereto, the at least one laser beam source 3 can include a pulse duration adapter 23 and a wavelength selector 24.

The treatment apparatus 1 can also comprise at least two of the laser beam sources 3, which can provide respective pulse durations and/or wavelengths. The control device 14 can be configured to adjust the respective one of the at least two laser beam sources 3, which is configured to output the pulse duration 4 to be adjusted and/or the wavelength 5 to be provided.

The treatment apparatus 1 can be configured for performing corneal cuts as the eye treatment method. The pulse duration 4 to be adjusted, the wavelength 5 to be adjusted as well as the numerical aperture 10 to be adjusted, which are required hereto, can be preset in at least one first eye treatment configuration 16 of the eye treatment configurations 12. For performing corneal cuts, a pulse duration of the laser beam between 100 fs and 1 ps is preset in the at least one first eye treatment configuration 16 of the eye treatment configurations 12. According to the at least one first eye treatment configuration 16 of the eye treatment configurations 12, the numerical aperture, which is to be provided by the transfer optics, is set to a value between 0.3 and 1.0. By the first one of the eye treatment configurations 16, the wavelength 5 of the laser beam to be adjusted can be adjusted to a value between 380 nm and 1200 nm for corneal cuts.

The treatment apparatus 1 can be configured for performing eye treatment methods for laser-induced refractive index change (URIC) and/or eye treatment methods for laser-assisted refractive index shaping (RIS). The pulse duration 4 to be adjusted, the wavelength 5 to be adjusted as well as the numerical aperture 10 to be adjusted, which are required hereto, can be preset in at least one second eye treatment configuration 17 of the eye treatment configurations 12. For performing the eye treatment methods, a pulse duration of the laser beam between 10 fs and 150 fs is preset in the at least one second eye treatment configuration 17 of the eye treatment configurations 12. According to the at least one second eye treatment configuration 17 of the eye treatment configurations 12, the numerical aperture 10 to be adjusted, which is to be provided by the transfer optics 6, is set to a value between 0.18 and 0.35. By the second eye treatment configuration 17, the wavelength 5 of the laser beam 2 to be adjusted can be preset to a value between 380 nm and 1200 nm for eye treatment methods for laser-induced refractive index change and/or eye treatment methods for laser-assisted refractive index shaping.

The treatment apparatus 1 can be configured for performing eye treatment methods for corneal cross linking. The pulse duration 4 to be adjusted, the wavelength 5 to be adjusted as well as the numerical aperture 10 to be adjusted, which are required hereto, can be preset in at least one third eye treatment configuration 18 of the eye treatment configurations 12. For performing the eye treatment methods, the pulse duration 4 of the laser beam to be adjusted is preset between 20 fs and 200 fs in the at least one third eye treatment configuration 18 of the eye treatment configurations 12. According to the at least one third eye treatment configuration 18 of the eye treatment configurations 12, the numerical aperture 10 to be adjusted, which is to be provided by the transfer optics 6, is set to a value between 0.05 and 0.25. By the third eye treatment configuration 18, the wavelength 5 of the laser beam 2 to be adjusted can be preset to a value between 360 nm and 950 nm for eye treatment methods for corneal cross linking.

The treatment apparatus 1 can be configured for performing eye treatment methods for treating presbyopia or for performing eye treatment methods for treating vitreous body destructions, in particular vitreous floaters. The pulse duration 4 to be adjusted, the wavelength 5 to be adjusted as well as the numerical aperture 10 to be adjusted, which are required hereto, can be preset in at least one fourth eye treatment configuration 19 of the eye treatment configurations 12. For performing the eye treatment methods, the pulse duration 4 of the laser beam 2 to be adjusted is preset between 300 fs and 1 ps in the at least one fourth eye treatment configuration 19 of the eye treatment configurations 12. According to the at least one fourth eye treatment configuration 19 of the eye treatment configurations 12, the numerical aperture 10 to be adjusted, which is to be provided by the transfer optics 6, is set to a value between 0.05 and 0.15. By the fourth eye treatment configuration 19, the wavelength 5 of the laser beam 2 to be adjusted can be preset to a value between 760 nm and 1500 nm for eye treatment methods for treating presbyopia or for performing eye treatment methods for treating vitreous destructions, in particular vitreous floaters.

The treatment apparatus 1 can be configured for performing eye treatment methods for tissue ablations on the cornea. The pulse duration 4 to be adjusted, the wavelength 5 to be adjusted as well as the numerical aperture 10 to be adjusted, which are required hereto, can be preset in at least one fifth eye treatment configuration 20 of the eye treatment configurations 12. For performing the eye treatment methods, the pulse duration 4 of the laser beam 2 to be adjusted is preset between 100 fs and 1 ps in the at least one fifth eye treatment configuration 20 of the eye treatment configurations 12. According to the at least one fifth eye treatment configuration 20 of the eye treatment configurations 12, the numerical aperture 10 to be adjusted, which is to be provided by the transfer optics 6, is set to a value between 0.00 and 0.10. By the fifth eye treatment configuration 20, the wavelength 5 of the laser beam 2 to be adjusted can be preset to a value between 150 nm and 230 nm for eye treatment methods for tissue ablations on the cornea.

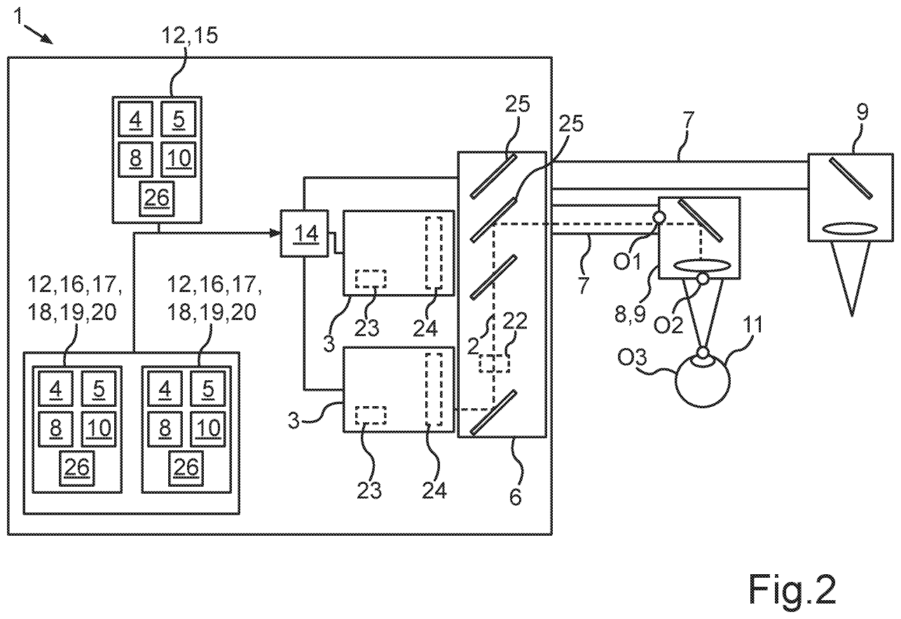

FIG. 2 shows a schematic representation of a further embodiment of the treatment apparatus. The treatment apparatus 1 can comprise two of the beam exit devices 9. The two beam exit devices 9 can be connected by respective optical paths 21. It can be provided that the beam exit devices 9 can be associated with respective eye treatment methods. The beam exit device 8 to be adjusted can be preset by the respective eye treatment configurations 12. The control device can be configured to adjust the beam exit selector 25 of the transfer optics 6 such that the laser beam 2 is fed to the beam exit device 8 to be adjusted by the beam transfer optics 6. The treatment apparatus 1 can comprise two laser beam sources 3, which can be configured for emitting respective laser beams 2. The wavelengths and/or pulse durations to be adjusted by the two laser beam sources 3 can differ from each other such that the control device has to adjust the laser beam source 3 of the laser beam sources 3 for emission of the wavelength to be adjusted and the pulse duration to be adjusted, which is provided for the respective values.

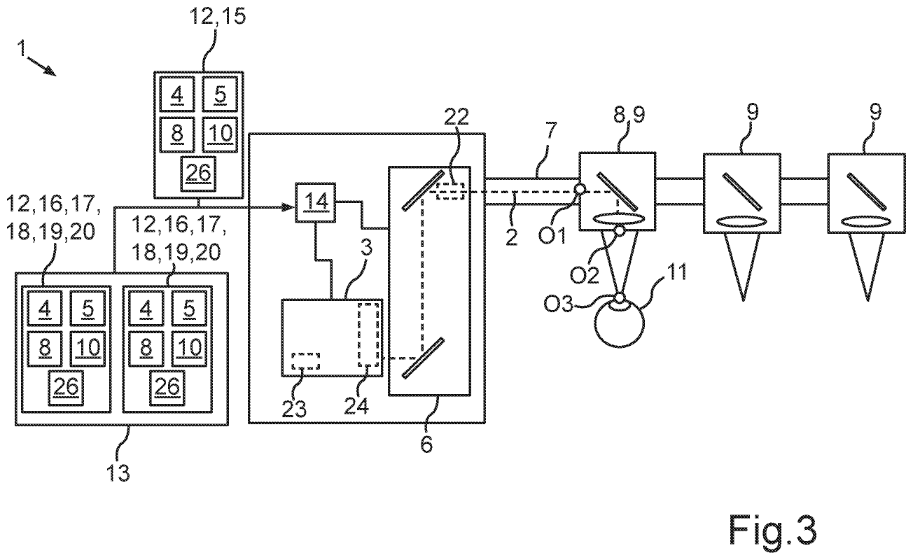

FIG. 3 shows a schematic representation of a further embodiment of the treatment apparatus. The embodiment of the treatment apparatus 1 can for example comprise three beam exit devices 9, which can be provided for performing respective eye treatments. The beam exit devices 9 can be connected to each other via an optical path. The control device 14 can be configured to adjust the beam exit selector 25 of the transfer optics 6 to feed the laser beam 2 to the corresponding beam exit device 9.

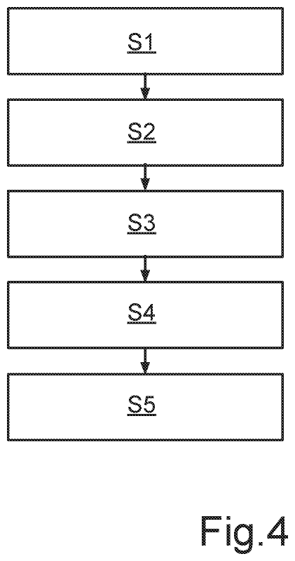

FIG. 4 shows a schematic representation of a sequence of a method. The method can be performed by the control device 14. The control device 14 is configured to perform a method. The above cited advantages arise. The control device can for example be configured as a control chip, control appliance or application program (“app”). The control device 14 can preferably comprise a processor device and/or a data storage. By a processor device, an appliance or an appliance component for electronic data processing is understood. For example, the processor device can comprise at least one microcontroller and/or at least one microprocessor. Preferably, a program code for performing the method according to the invention can be stored on the optional data storage. The program code can then be configured, upon execution by the processor device, to cause the control device 14 to perform one of the above-described embodiments of one or both methods according to the invention.

In a step S1, the preset eye treatment configuration 15 can be received from the storage device 13 and/or be retrieved from the storage device 13 by the control device of the treatment apparatus 1.

In a further step S2, the predetermined eye treatment configuration 15 can be read out and the pulse duration 4 to be adjusted can be adjusted in the laser beam source 3 as well as the numerical aperture 10 to be adjusted can be adjusted in the transfer optics 6 by the control device 14.

In a step S3, the wavelength 5 to be adjusted can be adjusted in the laser beam source 3 by the control device 14.

In a step S4, the beam exit device 9 to be adjusted can be adjusted in the transfer optics 6 by the control device 14.

In a step S5, the energy 26 of a laser pulse to be adjusted can be adjusted in the laser beam source 3 by the control device 14.

### LIST OF REFERENCE CHARACTERS

1 Treatment apparatus
2 Laser beam
3 Laser beam source
4 Pulse duration to be adjusted
5 Wavelength to be adjusted
6 Transfer optics
7 Optical path
8 Beam exit device to be adjusted
9 Beam exit device
10 Numerical aperture to be adjusted
11 Eye
12 Eye treatment configuration
13 Storage device
14 Control device
15 Predetermined eye treatment configuration
16 First eye treatment configuration
17 Second eye treatment configuration
18 Third eye treatment configuration
19 Fourth eye treatment configuration
20 Fifth eye treatment configuration
21 Optical path
22 NA adapter
23 Pulse duration adapter
24 Wavelength selector
25 Beam exit selector
26 Energy to be adjusted
O1 Point
O2 Point
O3 Point
S1-S5 Method steps

1. A treatment apparatus for an eye treatment comprising:
at least one laser beam source configured for emission of a laser beam;
at least one beam exit device, which is configured to direct the laser beam to an eye to be treated;
at least one transfer optics, which is configured to feed the laser beam along a respective optical path to the at least one beam exit device; and
a control device, which is configured to retrieve a predetermined eye treatment configuration from a storage device, on which multiple eye treatment configurations are stored, to adjust the at least one laser beam source for generating the laser beam of a pulse duration to be adjusted according to the predetermined eye treatment configuration of the eye treatment configurations, and to adjust the at least one transfer optics for providing a predetermined numerical aperture of the transfer optics according to the predetermined eye treatment configuration.

2. The treatment apparatus according to claim 1, wherein the treatment apparatus includes a laser beam source.

3. The treatment apparatus according to claim 1, wherein the control device is configured to adjust the at least one laser beam source for generating the laser beam of a wavelength to be adjusted according to the predetermined eye treatment configuration.

4. The treatment apparatus according to claim 1, wherein in at least one first eye treatment configuration of the eye treatment configurations, the pulse duration of the laser beam to be adjusted is between 100 fs and 1 ps, and the numerical aperture of the transfer optics to be adjusted is between 0.3 and 1.0.

5. The treatment apparatus according to claim 4, wherein in the at least one first eye treatment configuration, a wavelength of the laser beam to be adjusted is between 380 nm and 1200 nm.

6. The treatment apparatus according to claim 1, wherein in at least one second eye treatment configuration of the eye treatment configurations, the pulse duration of the laser beam to be adjusted is between 10 fs and 150 fs, and the numerical aperture of the transfer optics to be adjusted is between 0.18 and 0.35.

7. The treatment apparatus according to claim 6, wherein in the at least one second eye treatment configuration, a wavelength of the laser beam to be adjusted is between 380 nm and 1200 nm.

8. The treatment apparatus according to claim 1, wherein in at least one third eye treatment configuration of the eye treatment configurations, the pulse duration of the laser beam to be adjusted is between 20 fs and 200 fs and the numerical aperture of the transfer optics to be adjusted is between 0.05 and 0.25.

9. The treatment apparatus according to claim 8, wherein in the at least one third eye treatment configuration, a wavelength of the laser beam to be adjusted is between 360 nm and 950 nm.

10. The treatment apparatus according to claim 1, wherein in at least one fourth eye treatment configuration of the eye treatment configurations, the pulse duration of the laser beam to be adjusted is between 300 fs and 1 ps and the numerical aperture of the transfer optics to be adjusted is between 0.05 and 0.15.

11. The treatment apparatus according to claim 10, wherein in the at least one fourth eye treatment configuration, a wavelength of the laser beam to be adjusted is between 760 nm and 1500 nm.

12. The treatment apparatus according to claim 1, wherein in at least one fifth eye treatment configuration of the eye treatment configurations, the pulse duration of the laser beam to be adjusted is between 100 fs and 1 ps and the numerical aperture of the transfer optics to be adjusted is between 0 and 0.1.

13. The treatment apparatus according to claim 12, wherein in the at least one fifth eye treatment configuration, a wavelength of the laser beam to be adjusted is between 150 nm and 230 nm.

14. The treatment apparatus according to claim 1, wherein:
the treatment apparatus includes at least two of the beam exit devices, and
the control device is configured to adjust the at least one transfer optics for feeding the laser beam to a beam exit device to be adjusted according to the predetermined eye treatment configuration.

15. The treatment apparatus according to claim 1, wherein the control device is configured to adjust the at least one laser beam source for generating the laser beam of an energy, to be adjusted, of a laser pulse of the laser beam according to the predetermined eye treatment configuration.

16. A method for controlling the treatment apparatus according to claim 1, by the control device, comprising at least the steps of:
adjusting the at least one laser beam source for generating the laser beam of the pulse duration to be adjusted according to the predetermined eye treatment configuration; and
adjusting the at least one transfer optics for providing the numerical aperture to be adjusted according to the predetermined eye treatment configuration.

17. The method according to claim 16, comprising at least the further step of:
adjusting the at least one laser beam source for generating the laser beam of the wavelength to be adjusted according to the predetermined eye treatment configuration.

18. The method according to claim 16, comprising at least the further step of:
adjusting the at least one transfer optics for feeding the laser beam to the beam exit device to be adjusted according to the predetermined eye treatment configuration.

19. The method according to claim 16, comprising at least the further step of:
adjusting the at least one laser beam source for generating the laser beam of the energy, to be adjusted, of a laser pulse of the laser beam according to the predetermined eye treatment configuration.

20. A computer program comprising commands, which cause the treatment apparatus according to claim 1 to execute the steps of:
adjusting the at least one laser beam source for generating the laser beam of the pulse duration to be adjusted according to the predetermined eye treatment configuration; and
adjusting the at least one transfer optics for providing the numerical aperture to be adjusted according to the predetermined eye treatment configuration.

21. A computer-readable medium, on which the computer program according to claim 20 is stored.

22. A storage device, on which the eye treatment configurations according to claim 1 is stored.
